(19) United States
(12) Patent Application Publication
(10) Pub. No.: US 2024/0335575 A1
(43) Pub. Date: Oct. 10, 2024

(54) METHOD AND APPARATUS FOR INACTIVATING PATHOGENS IN UNITS OF WHOLE BLOOD USING SUPERPARAMAGNETIC NANOPARTICLES COATED WITH CHEMILUMINESCENCE REAGENTS AND BROAD-SPECTRUM ANTI-VIRAL THERAPEUTICS

(71) Applicant: Trevor Percival Castor, Arlington, MA (US)

(72) Inventor: Trevor Percival Castor, Arlington, MA (US)

(73) Assignee: Aphios Corporation, Arlington, MA (US)

(21) Appl. No.: 18/034,941

(22) PCT Filed: Oct. 20, 2021
(86) PCT No.: PCT/US2021/055914

§ 371 Date: Apr. 20, 2024

(30) Foreign Application Priority Data
Oct. 20, 2021 (US) 63094337

(51) Int. Cl. A61L 2/00; A61K 41/10; A61L 101/44

(52) U.S. Cl. CPC A61L 2/0082; A61K 41/10; A61L 2101/44; A61L 2202/22

(57) ABSTRACT

A method and apparatus for reducing or inactivating pathogens in units of whole blood. A plurality of superparamagnetic nanoparticles (SPN) is coated with a mixture of chemiluminescence light-generating compounds and photodynamic broad-spectrum anti-viral compounds, and the mixture in introduced into a bag of whole blood. A rapidly-changing electromagnetic field is applied to the bag to cause uniform distribution of the nanoparticles within the whole blood throughout all regions of the blood bag, including the opaque interior of the bag. The blood is processed for a predetermined processing time period, during which the chemiluminescence light activates the broad-spectrum antiviral capacity of the photodynamic compounds to achieve reduction or inactivation of pathogens throughout the blood bag. After the processing time is elapsed, the nanoparticles are removed from the processed blood by a magnetic field. The processed blood may be washed by conventional means, to remove residual reagents, and transferred into a new, sterile blood bag.

### FIELD OF INVENTION

The present invention relates to pathogen reduction and inactivation in units of whole blood using superparamagnetic nanoparticles (SPN) coated with chemiluminescence reagents and broad-spectrum antiviral therapeutics (CAT).

### REFERENCES TO OTHER PATENTS

This application discloses a number of improvements and enhancements to the anti-viral complexes and methods disclosed in U.S. Pat. No. 7,027,524 to Castor et al., which is hereby incorporated by reference in its entirety.

### BACKGROUND OF THE INVENTION

A number of emerging viruses such as SARS-CoV-2, Zika, West Nile, SARS Coronavirus, the Mexican swine flu and other potential bioterrorism pathogens like smallpox are not conventionally screened for, but are of concern to the safety of the human blood supply chain. The causes of the more rapid emergence and spread of some of these “killer” pathogens are not entirely known, but are thought to be caused by some combination of deforestation with urbanization of wild virus habitats, evolutionary mutations and rapid global travel.

The periodic emergence of severe viral respiratory infections (e.g., SARS, MERS and COVID-19), the rapid spread of the Zika virus, which can have a significant impact on neurological disorders in unborn fetuses and potentially adults, the recent outbreaks of the extremely virulent Ebola virus and potentially pandemic strains of influenza (e.g., H5N1), and the worldwide AIDS epidemic have highlighted a persistent concern in the health-care community—the need for effective pathogen inactivation and removal techniques for human blood, plasma and plasma-derived products.

Even with the increased emphasis on screening of donors and testing for pathogens, the risk of transmission of infection by blood products is currently estimated to be 1 in 205,000 for hepatitis B virus (HBV), 1 in 2 million for hepatitis C virus (HCV) and 1 in 2 million for HIV (NHLBI, NIH, 2016). These numbers are significantly higher when disaggregated by region or socio-demographic clusters within and outside of the US. In addition, viruses such as human T-cell lymphotropic viruses (HTLV) types I and II, human parvovirus B19, cytomegalovirus (CMV) and Epstein-Barr virus (EBV) pose potential risks following the transfusion of blood and blood components. Viruses of major concern as pathogens in human blood plasma include such as human parvovirus B19 and the hepatitis A, B and C viruses (non-enveloped viruses), and the enveloped viruses like human immunodeficiency viruses HIV-1 and HIV-2, and herpes viruses (CMV, EBV, HHV-6, HHV-7, HHV-8). For most of these viruses, the short period between initial infection and seroconversion presents a window of significant transfusion risk as routine clinical tests are not robust to detect the virus. CMV seroprevalence for example may range from 40%-100% depending on setting, and establishes as a life-long latent infection with severe morbidity to patients.

Annually, an estimated 3.8 million Americans are transfused with 28.2 million blood components derived from 12.8 million units of blood donated by apparently healthy volunteers. A rigorous scrutiny of blood donors and screening of donated blood for various serological markers have significantly reduced the mortality and morbidity due to transfusion-transmitted infectious diseases. Some enzyme immunoassays used for routine screening may detect viral antigens or antibodies, but not the infectious agents themselves. Moreover, reliance on patient disclosure and risk assessment questionnaires has previously failed to correctly identify virus infected blood donors (CDC, 2010). Thus, there could be an asymptomatic window period of infectivity responsible for a residual risk of post-transfusion infection.

Whereas conservative data estimates 1 in every 1.5 million are at a risk of transfusion transmitted infection (TTI) in the U.S (CDC, 2010), increasing globalization of public health increases the risk of TTI to Americans substantially. Moreover, the high frequency episodes of emergent and seasonal infectious diseases like Ebola, Zika, MERS, SARS, West Nile virus (WNV), and rare yet fatal blood borne infectious agents like Prion Diseases and Variant Creutzfeldt-Jakob Disease (vCJD), continue to cause localized and global-level public health risk from TTI. Combined, these risks supersede the risks reported within individual country borders. In many developing countries, the risk of being transfused with donor blood infected with at least one pathogen (including HIV, HCV & HBV) is substantially high, ranging from 9.5%-21.1% in Ethiopia to 1.2%-15% in China.

These risks and those of emergent infectious epidemics like Zika, follow regional and socio-demographic trends, from which developed countries cannot be precluded. The variable but important global burden of TTI means new processes and products that make blood products safer will not only benefit public health, but would be significant commercial innovations.

A number of approaches have been employed for the inactivation or removal of viruses in human plasma, harnessing therapeutic proteins derived from human plasma and preparation of recombinant biologics. These include heating or pasteurization; solvent-detergent technique; Ultraviolet (UV) irradiation; chemical inactivation utilizing hydrolyzable compounds such as β-proprionolactone and ozone; and photochemical decontamination using synthetic psoralens. The major problems with pasteurization include long pasteurization times, deactivation of plasma proteins and biologics, and the use of high concentrations of stabilizers that must be removed before therapeutic use. The solvent-detergent (SD) technique is quite effective against lipid-coated or enveloped viruses such as HIV, HBV and HCV, but is ineffective against protein-encased or non-enveloped viruses such as HAV and parvovirus B19. The solvent-detergent technique is also burdened by the need to remove residual organic solvents and detergents before therapeutic use.

The photochemical-psoralen method, while quite effective with a wide range of viruses, is burdened by potential residual toxicity of photoreactive dyes and other potentially carcinogenic or teratogenic compounds. However, the Cerus Intercept method has been recently approved by the FDA for the viral clearance of human plasma, red blood cells and platelets. Intercept is effective against both enveloped and some but not all non-enveloped viruses; HAV, HEV, B19, and Poliovirus are resistant to the Cerus inactivation process. Thus, current approaches are not always effective against a broad spectrum of human and animal viruses, are sometimes encumbered by process-specific deficiencies, and often result in denaturation of the target biologics.

To reduce transfusion transmitted infection, there is a need for an effective, general purpose, integrated solution for broad-spectrum purifying of whole blood by the reduction and inactivation of pathogens, especially when the identity of the pathogens in the blood may be unknown or not predetermined. There is a need for a method of removing pathogens from whole blood that avoids the drawbacks and disadvantages of prior methods, as stated above.

### SUMMARY OF THE INVENTION

The present invention is an integrated pathogen reduction technology for whole blood, using superparamagnetic nanoparticles (SPN) coated with chemiluminescence reagents and broad-spectrum antiviral therapeutics (CAT), providing minimal reduction in biological integrity and potency in order to reduce the risk of transfusion-mediated transmission of known as well as unknown pathogens and potential bioterrorism threats, and eliminate or minimize the need for downstream pathogen clearance steps for human blood products including plasma, red blood cells and platelets.

In one aspect, the invention uses superparamagnetic nanoparticles (SPN) coated with chemiluminescence reagents and broad-spectrum antiviral therapeutics (CAT) to inactivate pathogens in units of whole blood. In another aspect, a changing magnetic field causes rapid mixing of the nanoparticles into the whole blood to minimize diffusion-limitations and shorten processing times. In a further aspect, a magnetic field is used to remove the nanoparticles and chemical reagents from the whole blood after pathogen inactivation.

Chemiluminescence reagents on nanoparticles interact with specific enzymes in solution to produce an in situ light signal for activate photodynamic broad-spectrum anti-viral compounds, which in turn inactivate viruses and other pathogens in the blood bag including the internal opaque areas of the blood bag. In another embodiment, Ex situ light sources are also used to activate chemiluminescence reagents in peripheral regions of the blood bag, which do not normally receive illumination.

In another aspect, the reagents can be placed in the blood collection bag prior to blood donation. The bag is then placed between the poles of a magnet in a custom designed and built SPN-CAT processing unit. After processing, the whole blood is transferred to a fresh bag for storage and/or further processing. Optionally, prior to transfer, the pathogen cleared blood is washed to reduce the concentrations of any residual reagents.

Presently, there is no commercially available, FDA-approved technology for the pathogen reduction of whole blood. We have previously demonstrated that hypericin in the presence of a “molecular flashlight” can significantly inactivate HIV in the dark (10⁶ TCID₅₀) in cell culture media in vitro without harm to these cells [Castor et al., US patent, 2006]. We have also established CAT compositions to inactivate >3.82 logs of Bovine Viral Diarrhea Virus (BVDV), a nonenveloped virus often used as a surrogate for Hepatitis C; and 2.4 logs of the nonenveloped Human Adenovirus-2 (HAd-2) in unwashed RBCC samples. The optimization of CAT when combined with SPN is designed to provide broad-spectrum pathogen reduction of both enveloped and nonenveloped viruses, bacteria and parasites. The technology offers unique advantages not achievable by currently available competing products like that of SD and the Cerus Intercept.

SPN-CAT technology is applicable to both pooled human plasma and units of plasma. The technology changes current paradigms by eliminating or minimizing the need for downstream pathogen clearance steps for human blood products including plasma, red blood cells and platelets. This technology ensures a blood supply that is safe from emerging and unknown pathogens and bioterrorism threats. The potential impact of a generally-applicable pathogen reduction technology for both enveloped and non-enveloped viruses, and emerging pathogens with high retention of biological activity is very significant. The technology could be very impactful in developed countries and in hot zones for both the rapid virus clearance of pooled and units of whole blood.

These and other features, aspects and advantages of the present teachings will be better understood with reference to the following drawings, description, examples and appended claims.

### BRIEF DESCRIPTION OF THE DRAWINGS

FIG. 1 is a schematic representation of chemiluminescence-directed inactivation of COVID-19;

FIG. 2 is a graph showing the inhibition of HIV-1_{IIIB} growth in virally infected cultures by the concerted action of hypericin, chemiluminescent substrate and alkaline phosphatase.

FIG. 3 schematically illustrates the SPN-CAT whole blood Processing unit of the present invention;

FIG. 4 schematically illustrates the SPN-CAT whole blood electrical circuitry of the present invention; and

FIG. 5 is a flow chart showing the process flow of removing pathogens from whole blood, according to the present invention.

### DETAILED DESCRIPTION OF THE PREFEFFED EMBODIMENTS

The present invention encompasses the following features: (i) the use of chemiluminescence for the in situ generation of light to activate the antiviral capacity of photodynamic compounds in the opaque interior of whole blood bags; (ii) the use of an ex situ light source to activate the antiviral capacity of photodynamic compounds in the periphery of whole blood bags; (iii) chemistry to bridge and enhance the light generated; (iv) broad-spectrum antiviral compounds; (v) coated superparamagnetic nanoparticles for chemiluminescent compounds and broad-spectrum antiviral therapeutics; (vi) varying magnetic fields for mixing of coated nanoparticles with whole blood; (vii) separation of coated nanoparticles at the end of the pathogen reduction process; (viii) use of conventional blood bags and technology for pathogen reduction of whole blood; and (ix) if necessary, washing of the pathogen reduced whole blood prior to transfer into a new, sterile blood bag.

Chemiluminescence-directed antiviral activities of natural and synthesized light-sensitive compounds can be effective in combating a broad range of viral infections. The phenomenon of hypericin-induced viral inactivation has been described in the literature for several decades. Briefly, it has been established that even low concentrations of hypericin and some hypericin-related compounds inactivate most enveloped viruses, including HIV in the absence of significant in vitro cytotoxicity. Apart from inherent phototoxicity, which is neutralized when hypericin is light activated, the benign toxicity profile should be expected for hypericin since it is a major component in St. John's Wort extracts. Unfortunately, the therapeutic use of hypericin for antiviral treatment has been precluded by the major requirement for its action, exposure to visible light.

The efficiency of hypericin-induced light-mediated viral inactivation is so high that even relatively short exposure times, which have occurred during routine tissue culture infection procedures, were sufficient for nearly complete inactivation of the exposed virus, notably HIV and other retroviruses. Upon the realization of this light exposure requirement, it has been shown that fluorescent light provides an even higher degree of hypericin anti-viral activity than visible light, rendering non-infective over 10⁶ TCID₅₀ of HIV. On the contrary, if the virus is treated with hypericin in complete darkness, then the viricidal effects are minimal, if at all detectable.

Obviously, one should not expect any benefits from hypericin administration to patients afflicted by viral diseases since there is no light inside the organism. Despite this reasonable assumption, pilot studies of hypericin's benefits for HIV and hepatitis C-infected individuals have been performed with the predictable negative result. The main reason for conducting these trials was hypericin's extremely high anti-viral activity in vitro and its advantageous safety profile. At the same time, hypericin was tested for light-induced inactivation of viruses in blood-related products and this technology has attained a high degree of efficiency.

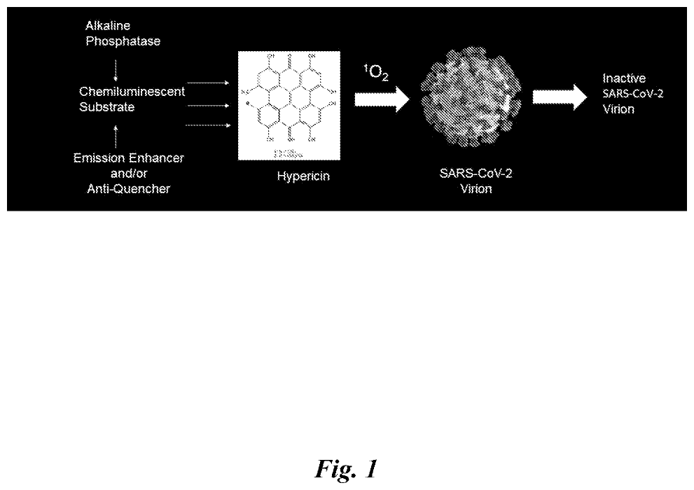

In order to generate an in situ light source, we have developed and patented a “molecular flashlight” that turns on when novel chemiluminescent substrates are combined with enzymes such as alkaline phosphatase and emission enhancers or anti-quenchers. [U.S. Pat. No. 7,027,524 to Castor et al., 2006]. Inactivation of viral pathogens, such as SARS-CoV-2, the etiologic agent that causes COVID-19, according to the present invention, is illustrated in FIG. 1.

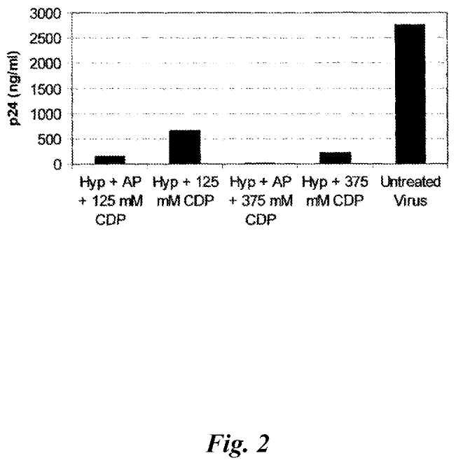

The combined use of hypericin, a light-emitting substrate, and an emission enhancer and light-generating enzyme is used to achieve significant inactivation of enveloped viruses such as HIV-1, as shown in FIG. 2. The present invention uses an enzyme normally present inside an organism, namely, alkaline phosphatase and highly active light-emitting substrates, which provide for long and sustained light emission, a specific wavelength peak of emission and an anti-quencher such as CDP-Star for dramatically lengthening chemiluminescence duration.

Hypericin treatment at 40 μg/mL with either luciferase at doses of 0.16, 0.32 or 0.80 μM or with CDP-Star at doses of 0.1 or 1 mM completely inactivated the enveloped virus BVDV in spiked Red Blood Cell Concentrates (RBCC). Hypericin at 40 μg/mL with either luciferase at 0.16 μM or CDP-Star at 0.1 mM showed very little impact on cytotoxicity, interference, RBC morphology and integrity.

Hypericin has absorbance peaks at 565 nm and 604 nm in PBS. Action of alkaline phosphatase on CDP-Star results in chemiluminescence with a peak emission at 475 nm. Sulforhodamine 101 has an absorbance peak at 586 nm and emission peak at 605 nm. A bridge compound that absorbs at 475 nm and emits at 585 nm would enhance the emission from sulforhodamine at 605 nm. Doxorubicin is suitable since it has an absorbance peak at 470 nm and an emission peak at 585 nm. Thus, the three compounds acting in concert result in maximum emissions at wavelengths that overlap the absorbance peaks of hypericin, resulting in a higher level of hypericin activation and more efficient viral inactivation.

Doxorubicin, an FDA approved anticancer drug, is a cytotoxic anthracycline antibiotic isolated from cultures of Streptomyces peucetius var. caesius. Doxorubicin binds to nucleic acids, presumably by specific intercalation of the planar anthracycline nucleus with the DNA double helix. Doxorubicin and its derivatives have known broad-spectrum antiviral, antimicrobial and anti-parasite properties. However, doxorubicin is known to have high toxicities including cardiac toxicity, and ability to reactivate Hepatitis B virus. Thus, the use of doxorubicin as a light enhancement bridge and a broad-spectrum antiviral is not recommended for an integrated pathogen reduction technology without a dependable way to ensure its removal. This is achieved by utilizing superparamagnetic nanoparticles coated with doxorubicin that are removed from the whole blood with a magnet. Alternative bridging compounds and/or broad-spectrum anti-pathogen therapeutics can be utilized.

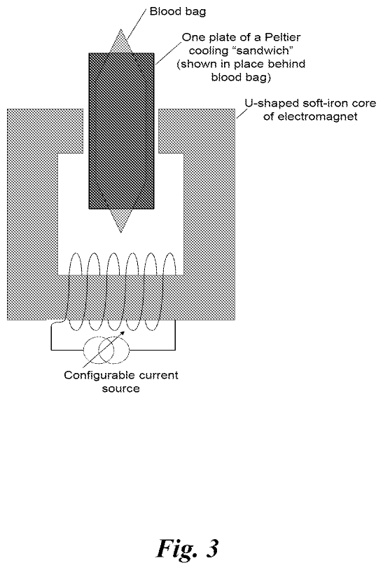

As shown in FIG. 3, magnetic nanoparticles (MNPs) are being used as image contrast probes, hydrothermal agents, magnetic-guide vectors and drug delivery carriers. The main advantages of using MNPs for such purposes include easy preparation, small sizes (>30 nm), chemical functionalization, biocompatibilities and stabilities, efficient drug conjugation and superior magnetic responsiveness (FIG. 3).

The most widely used systems in biological settings are MNPs made of iron oxides (Fe3O4/Fe2O3) due to their well-known biocompatibilities. When the size of the MPN is below a critical value (˜ 30 nm), these nanoparticles behave like a giant paramagnetic atom with a single magnetic domain exhibiting superparamagnetic behavior. Superparamagnetic nanoparticles respond rapidly to an applied magnetic field with negligible residual magnetism away from the magnetic field and when the magnetic field is turned off or removed.

The present invention includes functionalizing MNPs with chemiluminescent reagents and antiviral therapeutics to facilitate their mixing with the whole blood and their removal after pathogen reduction; and resident or added enzymes such as alkaline phosphatase in solution state in order to induce low-level luminescence (in conjunction with a hypericin-substrate-enhancer complex), which is toxic to viruses but not endogenous cells.

In another aspect, the present invention on also includes varying magnetic fields for mixing of coated nanoparticles with whole blood; separation of coated nanoparticles at the end of the pathogen reduction process; use of conventional blood bags and technology for pathogen reduction of whole blood; and if necessary, washing of the pathogen reduced whole blood prior to transfer into a new, sterile blood bag.

The present invention is an integrated pathogen reduction technology for units of whole blood by utilizing superparamagnetic nanoparticles (SPN) coated with chemiluminescence reagents and broad-spectrum antiviral therapeutics (CAT).

Magnetic nanoparticle formulations are used for the delivery of hypericin, chemiluminescent substrates, anti-quenchers and select antiviral therapeutics of a broad-spectrum antiviral cocktail, and evaluate their paramagnetic removal. These formulations are optimized the inactivation of Bovine Viral Diarrhea Virus (BVDV), a surrogate for Hepatitis C, as a representative enveloped virus and the Human Adeno Type 2 (Had-2), a DNA virus, as a representative enveloped virus and select CAT components based on in vitro efficacy and cytotoxicity studies.

The process utilizes superparamagnetic nanoparticle formulations of chemiluminescent substrates, anti-quenchers and a select antiviral therapeutic. The process optimizes the inactivation of Bovine Viral Diarrhea Virus (BVDV), a surrogate for Hepatitis C, as a representative enveloped virus and the Human Adeno Type 2 (Had-2), a DNA virus, as a representative enveloped virus and select CAT components based on in vitro efficacy, cytotoxicity and interference studies. The chemical components of the CAT system consist of alkaline phosphatase and luciferase enzymes, photoactive compound hypericin, chemiluminescent substrates, emission enhancers (or anti-quenchers) such as CDP Star® and D-Luciferin and the broad-spectrum anti-pathogenic agent, doxorubicin.

Hypericin [C₃₀H₁₆O₈; Molecular Weight: 504.45; CAS Number: 548-04-9; Aphios Catalog No: APH-20013] is a naphthodianthrone, a red-colored anthraquinone-derivative.

CDP Star®: [C₁₈H₁₉Cl₂O₇Na₂P; Disodium 2-chloro-5-(4-methoxyspiro{1,2-dioxetane-3,2′(5′-chloro)tricyclo[3.3.1.13,7]decan}-4-yl)-1-phenyl phosphate; MW=495.2; CAS No. 160081-629; Sigma-Aldrich Catalog No. C0712].

D-Luciferin: [C₁₁H₈N₂O₃S₂; Firefly Luciferin 4,5-Dihydro-2-(6-hydroxy-2-benzothiazolyl)-4-thiazolecarboxylic acid; MW=280.3; CAS No.: 2591-17-5; Sigma-Aldrich Catalog No. L9504].

Emission Enhancers (or Anti-Quenchers): Tropix enhancers such as Sapphire™, Emerald™, Ruby™, Sapphire-II™, and Emerald-II™ enhancers are essential components of solution-based assays. Enhancers provide signal enhancement with minimal decay of light-emission kinetics, and allows shift of the wavelength of light emission.

Doxorubicin: [C₂₇H₂₉NO₁₁ (1S,3S)-3-glycoloyl-3,5,12-trihydroxy-10-methoxy-6,11-dioxo-1,2,3,4,6,11-hexahydrotetracen-1-yl 3-amino-2,3,6-trideoxy-α-L-lyxo-hexo-pyranoside; MW=543.525 g·mol⁻¹; CAS No. 23214-92-8].

Superparamagnetic Nanoparticles (SPN): Nanoparticle formulations of chemiluminescent substrates and antiviral therapeutics are manufactured using commercially-available functionalized iron nanoparticles (5-20 nm) [OCNIAZ20—PEG-azide functionalized; OCNIC52520—Carboxylic acid functionalized; and OCNIA52505—Amine functionalized; Sigma-Aldrich]. Formulations are purified by a combination of magnetism and washing and, if necessary, by size exclusion chromatography. Nanoformulations are tested by HPLC-RP-UV, UV-Vis and particle size analysis using photo correlation spectroscopy. Following treatment, SPNs are removed as thoroughly as possible through one or multiple rounds of magnetic removal and the SPN-free materials tested for functionality and/or residual infectivity.

Optimization of the pathogen reduction experiments are performed initially at bench scale (using the smallest possible volumes of components for the assays) on whole blood spiked with bovine viral diarrhea virus (BVDV) as the representative enveloped virus, a surrogate for Hepatitis C, and human adenovirus type 2 (HAd-2) as the representative non-enveloped virus. Viral inactivation studies are be performed using whole blood spiked with virus stocks in accordance with CPMP/FDA guidelines. The spiked blood is treated with multiple doses of photodynamic/chemiluminescent compound combinations. Design of experiments (DOEs) are performed to determine the optimum concentration ranges for each of the components by varying the concentrations of each component based on prior results. Appropriate positive and negative controls are used to determine the contribution of each of the components to viral inactivation.

Cytotoxicity and interference studies are performed per CPMP/FDA guidelines to calculate the reduction in viral titers due to these factors and exclude them from the calculated reduction factors resulting from the antiviral treatment. The antiviral compounds are removed from the system by washing the erythrocytes. Appropriate controls are kept to determine the contribution of washing to the viral reduction factors.

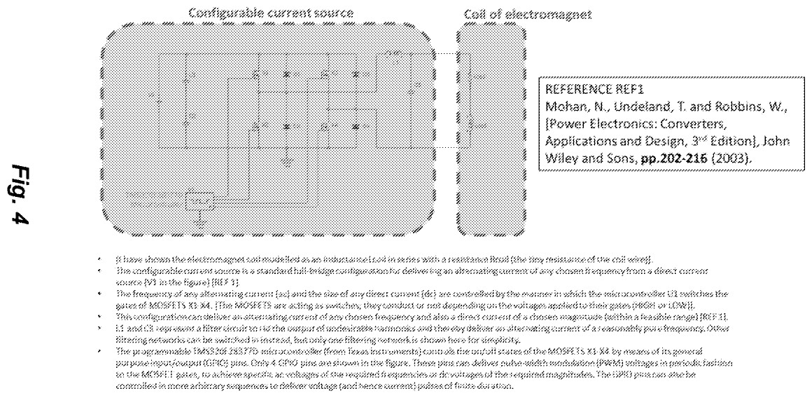

SPN-CAT Unit: The SPN-CAT processing unit is shown schematically in FIG. 3. The configurable current source is shown in FIG. 4. In this unit, the whole blood bag with chemiluminescence reagents and antiviral therapeutics are placed in the unit between a Peltier cooling device on the lateral side and between the poles of a U-shaped, soft-iron core electromagnet. Peltier cooling is designed to maintain the blood bag between 4° C. and 10° C. to maintain biological activity.

If electromagnetic removal is insufficient and/or there are residual reagents and/or toxicity in the pathogen reduced whole blood, inter-bag washing of the pathogen reduced whole blood will be performed with Haemonetics ACP 215 or equivalent.

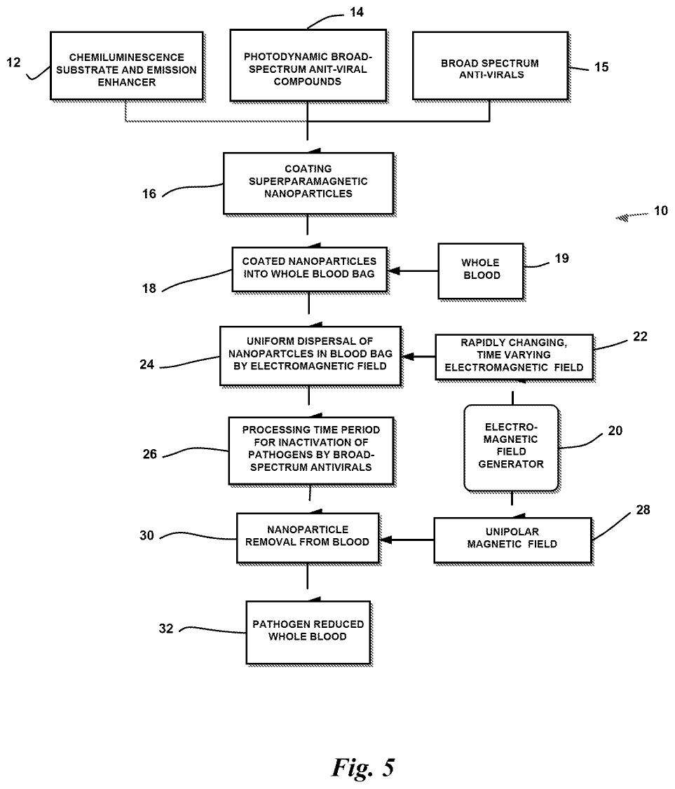

Turning now to FIG. 5, the method and apparatus of the present invention 10 is shown in flowchart form. The chemiluminescent substrate and emission enhancer 12 are combined with a photodynamic broad-spectrum antiviral compound 14, such as Hypericin, or a broad-spectrum antiviral 15 such as Doxorubicin. As previously discussed, Doxorubicin and its derivatives have known broad-spectrum antiviral, antimicrobial and anti-parasite properties. However, Doxorubicin is also known to have high toxicities, which require that it be fully removed from the whole blood after processing. Therefore, the most desirable broad-based antiviral compound under investigation is Hypericin, which has minimal toxic properties. The chemiluminescent substrate and emission enhancer 12 and the broad-spectrum antiviral compounds 14,15 are used to coat a plurality of superparamagnetic nanoparticles 16, which are introduced into a whole blood bag 18. It is within the scope of the present invention that the plurality of superparamagnetic nanoparticles 16 may be introduced to an empty blood bag before a blood donor supplies whole blood 19 the bag. It is also in the scope of the present invention that the plurality of superparamagnetic nanoparticles 16 may be introduced into a blood bag 18 which already contains whole blood 19 from a donor. The present invention will function equally well following either processing scenario.

When the coated nanoparticles 18 are mixed with whole blood 19, it is important that there be a uniform distribution or dispersal of the coated nanoparticles 18 throughout the whole blood 19. This is achieved by the SPN-CAT processing unit, which is shown in FIGS. 10 and 11. In addition to holding the blood bag at an optimal temperature by close proximity to a Peltier cooling device, the processing unit includes the elements of a soft-ion core electromagnet and controller, which is designated in the flowchart as the electromagnetic field generator 20. A rapidly changing, time-varying electromagnetic field is established in the blood bag 18, causing the coated nanoparticles to disperse uniformly 24 throughout the blood bag 18, including all opaque regions in the center of the blood bag 18, and peripheral regions at the comers. As an enhancement to this process, ex situ light sources may be used to activate chemiluminescence reagents in peripheral regions of the blood bag.

The time varying electromagnetic field 22 mixes the coated nanoparticles with the whole blood for a predetermined processing time 26, during which the pathogens in the blood are reduced or inactivated. At the end of the pathogen reduction process, the processed blood is exposed to a unipolar magnetic field 28, and the nanoparticles are removed from the pathogen reduced whole blood 30. As a further step, it is contemplated that the reduced pathogen blood 32 may be washed using conventional blood processing, to remove any residual reagents prior to being transferred into a new, sterile blood bag.

### EXAMPLES

### Example 1: Inhibition of HIV-1_{IIIB} Growth in Infected Cultures by a Concerted Action of Hypericin, Chemiluminescent Substrate and Alkaline Phosphatase

CEM-SS cells were infected with 13-20 TCID₅₀ of HIV-1_{IIIB}, and then incubated with hypericin (5 μmol), alkaline phosphatase (Calbiochem, 0.18 U) and chemiluminescent substrate CDP in the concentrations shown. Tissue culture media was replaced every 3-4 days. HIV replication was measured by the amount of p24 capsid protein in the culture media from day 7. The median data of three replicates are listed in Table 1 and shown in FIG. 2. The alkaline phosphatase-induced hypericin action is also effective against fully competent virus.

TABLE 1
Inhibition of HIV-1_{IIIB} Growth in Infected Cultures
by a Concerted Action of Hypericin, Chemiluminescent
Substrate and Alkaline Phosphatase

| HIV-1_{IIIB}-infected CEM-SS cells | p24 concentration (ng/ml) |
|---|---|
| treated with: | on day 7 post-infection |
| Hyp + AP + 125 μm CDP + dark | 161 |
| Hyp + 125 μm CDP + dark | 664 |
| Hyp + AP + 375 μm CDP + dark | 6 |
| Hyp + 375 μm CDP + dark | 219 |
| Untreated virus | 2766.7 |

### Example 2: Virucidal Effect of Chemiluminescence-Induced Hypericin Action in the Presence of Luminescence Enhancer

We evaluated the level of virucidal effect of chemiluminescence-induced hypericin action in the presence of a luminescence enhancer. 50 TCID₅₀ (1 ng of p24) of HIV-1ΔtatΔrev viral stocks were pre-treated with a mixture of hypericin (5 μmol, Hyp), luminescence substrate with Ruby™ enhancer (Ruby) and different doses of alkaline phosphatase (AP) for 2 hours at 37° C. and then used to infect CEM-TART cells. Cells culture media was exchanged every 3-4 days and samples for p24 analysis were taken at the same time. HIV replication was measured by amount of p24 capsid protein in the culture media. Sample treated with hypericin exposed to the light was a positive control of viral inhibition; unexposed samples (Hyp only) were used as negative control. These experiments were conducted in triplicate and showed good reproducibility and inactivation dependent on the concentration of the luminogenic enzyme.

### Example 3: BVDV Inactivation by Hypericin with Luciferase

The objective of this experiment was to demonstrate the inactivation of BVDV spiked into human RBCC by hypericin in the presence of chemiluminescence produced by the action of luciferase on luciferin in the presence of ATP. Ten different combinations of DMEM (—Ca⁺⁺, —Mg⁺⁺), RBCC, virus, hypericin, Luciferin, ATP and luciferase were prepared to all contain equivalent titers of BVDV calculated to be 6.48 log₁₀TCID₅₀/mL based on the titer of the original virus stock. The concentrations of hypericin tested were 0, 40 and 200 μg/mL. Luciferase was tested at 0, 0.8 and 4.0 μM. Luciferin and ATP were present at 80 and 800 μM respectively when luciferase was present and absent in the absence of luciferase. The different combinations tested are listed in Table 2.

The components were mixed and incubated at RT for 2 hours in the dark followed by titration of unwashed and washed samples as previously described. Washing rows of wells with dilutions 1 and 2 was performed 3 days post-infection. Final CPEs were read 10 days post-infection.

The various combinations tested, the titers obtained and the calculated VRFs compared to the titer of the untreated RBC control are listed in Table 2. No clotting of RBC was observed for any of the samples during the assay.

TABLE 2
Hypericin-Luciferase Inactivation of BVDV

| Sample | | | Luciferase | Unwashed | | Washed | | Combined |
|---|---|---|---|---|---|---|---|---|
| # | Description | Hypericin | System | Titer | VRF | Titer | VRF | VRF |
| 1 | HN-LN (RBC | No | No | 6.35 | 0.00 | 3.98 | 0.00 | 2.37 |
| | Control) | | | | | | | |
| 2 | HN-LL | No | Low | 6.23 | 0.12 | 3.73 | 0.25 | 2.62 |
| 3 | HN-LH | No | High | 6.48 | −0.13 | 3.10 | 0.88 | 3.25 |
| 4 | HL-LN | Low | No | 3.48 | 2.87 | <2.34 | >1.64 | >4.01 |
| 5 | HL-LL | Low | Low | 2.98 | 3.37 | <2.34 | >1.64 | >4.01 |
| 6 | HL-LH | Low | High | 3.23 | 3.12 | <2.34 | >1.64 | >4.01 |
| 7 | HH-LN | High | No | 3.10 | 3.25 | 2.48 | 1.50 | 3.87 |
| 8 | HH-LL | High | Low | 3.23 | 3.12 | 2.60 | 1.38 | 3.75 |
| 9 | HH-LH | High | High | 3.35 | 3.00 | 2.48 | 1.50 | 3.87 |
| 10 | Virus Control | No | No | 6.10 | NA | NA | NA | NA |
| | (VC) | | | | | | | |

Notes:
1. Hypericin: No = 0, low = 40 μg/mL, high = 200 μg/mL
2. Luciferase: No = 0, low = 0.8 μM, high = 4.0 μM
3. Luciferin and ATP were at 0.08 and 0.8 mM respectively for both Luciferase ‘high’ and ‘low’ but absent in ‘No’ luciferase.

There was no detectable inactivation of virus for washed samples in the absence of hypericin except for the sample treated with high dose of luciferase system where a VRF of 0.88 logs was seen. However, combined VRFs of 2.37 to 3.25 logs were seen for the three washed samples treated with no hypericin which could be explained by the reduction in titers from washing and storage at RT during the assay period.

VRFs in the range of 4 logs were seen for all hypericin treated washed samples—both low and high doses of hypericin. The highest VRF of 3.37 logs was seen for unwashed samples treated with low doses of both hypericin and luciferase. Low dose hypericin treatment with or without luciferase followed by washing resulted in complete elimination of the virus to undetectable levels, and appeared to be more effective than the high dose of hypericin, where residual virus was detectable in the washed samples. These results indicate that a combination of hypericin at 40 μg/mL and luciferase at 0.8 μM was the most effective in BVDV inactivation with end product washing.

### Example 4: BVDV Inactivation by Hypericin with 3 doses of CDP-Star

Eleven different combinations of DMEM (—Ca⁺⁺, —Mg⁺⁺), RBCC, virus, hypericin, CDP-Star and alkaline phosphatase were prepared to all contain equivalent titers of BVDV calculated to be 6.48 log₁₀ TCID₅₀/mL based on the titer of the original virus stock. The concentrations of hypericin tested were 0, 40 and 200 μg/mL. CDP-Star stock at 25 mM was diluted to obtain final concentrations of 0.1, 1 and 10 mM. Alkaline phosphatase was present at 1.6 μM for all samples except the RBC control. The different combinations tested are listed in Table 3.

The components were mixed and incubated at 37° C. for 2 hours in the dark followed by titration of unwashed and washed samples as previously described. Washing rows of wells with dilutions 1 and 2 was performed the next day. Final CPEs were read 2 weeks post-infection.

The various combinations tested, the titers obtained and the calculated VRFs compared to the titer of the untreated RBC control are listed in Table 3. During the assay procedure, complete hemolysis was seen for samples treated with 10 mM, partial hemolysis with 1 mM and no hemolysis with 0.1 mM CDP-Star as seen in the previous experiment.

TABLE 3
Hypericin - CDP-Star Inactivation of BVDV

| Sample | | | | Unwashed | | Washed | | Combined |
|---|---|---|---|---|---|---|---|---|
| # | description | Hypericin | CDP-Star | Titer | VRF | Titer | VRF | VRF |
| 1 | HN-CL | No | Low | 5.85 | −0.38 | 3.35 | 0.38 | 2.13 |
| 2 | HN-CM | No | Med | 5.73 | −0.25 | 3.35 | 0.38 | 2.13 |
| 3 | HN-CH | No | High | <2.34 | >3.14 | <2.34 | >1.39 | >3.14 |
| 4 | HL-CL | Low | Low | 3.85 | 1.63 | <2.34 | >1.39 | >3.14 |
| 5 | HL-CM | Low | Med | 4.10 | 1.38 | <2.34 | >1.39 | >3.14 |
| 6 | HL-CH | Low | High | 2.85 | 2.63 | <2.34 | >1.39 | >3.14 |
| 7 | HH-CL | High | Low | 4.73 | 0.75 | 2.23 | 1.50 | 3.25 |
| 8 | HH-CM | High | Med | 3.60 | 1.88 | <2.34 | >1.39 | >3.14 |
| 9 | HH-CH | High | High | 3.85 | 1.63 | <2.34 | >1.39 | >3.14 |
| 10 | HN-CN(RBC | No | No | 5.48 | 0.00 | 3.73 | 0.00 | 1.75 |
| | Control) | | | | | | | |
| 11-L | Virus control | No | No | 5.73 | NA | ND | NA | NA |
| | (VC) | | | | | | | |
| 11-R | Virus stock | No | No | 7.10 | NA | ND | NA | NA |
| | (VS) | | | | | | | |

Notes:
1. Hypericin doses (μg/mL): No = 0, Low = 40, High = 200
2. CDP-Star doses (mM): Low = 0.1, Med = 1, High = 10
3. Left half of plate 11 was used for virus control and right half for the virus stock used for spiking.

These results indicate that hypericin at 40 μg/mL and CDP-Star at 0.1 mM appears to be the most optimal combination for inactivation of BVDV.

### Example 5: BVDV Inactivation by Doxorubicin Alone

Two levels of doxorubicin (low and high), RBCC, virus and DMEM (—Ca⁺⁺, —Mg⁺⁺) were prepared to all contain equivalent titers of BVDV calculated to be 6.23 log₁₀ TCID₅₀/mL (logs) based on the titer of the original virus stock.

The components were mixed and incubated at 37° C. for 2 hours in the dark followed by titration of unwashed and washed samples as previously described. Washing rows of wells with dilutions 1 and 2 was performed the next day. Final CPE were read 2 weeks post-infection. Cells were also monitored for cytotoxicity from the treatment components other than the virus 1 day and 2 weeks post-infection as in previous experiments.

The various combinations tested, the titers obtained and the calculated VRFs compared to the mean titer of the untreated RBC controls are listed in Table 4. There was no hemolysis or clumping for any of the samples at the end of the treatment period.

TABLE 4
Doxorubicin Inactivation of BVDV

| Sample | | Doxo- | Unwashed | | Washed | | Combined |
|---|---|---|---|---|---|---|---|
| # | Description | rubicin | Titer | VRF | Titer | VRF | VRF |
| 1 | RBC | Low | 5.73 | −0.13 | 2.60 | 0.31 | 3.00 |
| | Control-1 | | | | | | |
| 2 | RBC | Low | 5.48 | 0.13 | 3.23 | −0.31 | 2.38 |
| | Control-2 | | | | | | |
| 3 | Dox-L | Low | 3.23 | 2.38 | 3.10 | −0.19 | 2.50 |
| 4 | Dox-H | Low | 3.10 | 2.50 | 3.10 | −0.19 | 2.50 |
| 5-a | Virus | High | 5.60 | NA | ND | NA | NA |
| | Control | | | | | | |
| | (VC) | | | | | | |
| 5-b | Virus | High | 6.60 | NA | ND | NA | NA |
| | Stock | | | | | | |
| | (VS) | | | | | | |

Notes:
1. Doxorubicin doses (mM): Low = 0.53, High = 1.6
2. Left half of plate 5 was used for virus control and right half for the virus stock used for spiking.

The results suggest that doxorubicin treatment alone has viral inactivation activity against BVDV as observed previously with HAd-2 virus. Even though no VRFs were obtained for washed RBCC it is possible that the CPE observed for washed RBCC treated with doxorubicin could have been from cytotoxicity of the drug and not from viral infection.

The detailed description set forth above is provided to aid those skilled in the art in practicing the present invention. However, the invention described and claimed herein is not limited in scope by the specific embodiments herein disclosed. The embodiments are intended as illustrations of several aspects of the invention. Any equivalent embodiments are intended to be within the scope of this invention. Various modifications of the invention in addition to those shown and described herein will become apparent to those skilled in the art from the foregoing description which do not depart from the spirit or scope of the present inventive discovery. Such modifications are also intended to fall within the scope of the appended claims.

What is claimed is:

1. A method for reducing or inactivating pathogens in units of whole blood, comprising: (a) coating a plurality of superparamagnetic nanoparticles (SPN) with a chemiluminescence substrate and emission enhancer and photodynamic broad-spectrum anti-viral compounds (CAT); (b) introducing the coated nanoparticles into a whole blood bag containing donor; (c) applying a rapidly-changing electromagnetic field to the blood bag to cause uniform distribution of the nanoparticles within the whole blood throughout all regions of the blood bag; (d) processing the blood for a predetermined processing time period, wherein the chemiluminescence-generated light activates the broad-spectrum antiviral capacity of the photodynamic compounds, and wherein the activated photodynamic compounds reduce or inactivate pathogens throughout the blood bag; and (e) after the processing time period, applying unipolar magnetic field to the processed blood to remove the nanoparticles.

2. The method of claim 1, further including the step of washing the pathogen reduced whole blood to remove residual reagents after, and transferring the processed blood into a new sterile blood bag.

3. The method of claim 1, wherein the photodynamic broad-spectrum antiviral compound is Hypericin.

4. The method of claim one wherein the broad-spectrum antiviral compound is Doxorubicin.

5. The method of claim 1 wherein the coated nanoparticles are introduced into the blood bag prior to receiving donor whole blood.

6. The method of claim 1 wherein the coated nanoparticles are introduced into the blood bag after receiving donor whole blood.

7. A processing unit to facilitate the removal of pathogens from whole blood contained in a blood bag; (a) a blood bag receptacle for holding the blood bag, said blood bag containing whole blood and magnetic nanoparticles coated with an antiviral reagent; (b) a Peltier cooling sandwich in proximity to the blood bag for maintaining the temperature of the blood bag at a predetermined temperature; and (c) an electromagnetic field generator, in proximity to the blood bag receptacle, for exposing the blood bag to a rapidly changing, time varying electromagnetic field, causing uniform dispersal of the magnetic nanoparticles throughout the whole blood; wherein pathogens are reduced or inactivated by the antiviral reagents.

8. The processing unit of claim 7, wherein the magnetic nanoparticles are removed from the pathogen reduced whole blood by unipolar magnetic field.

9. The processing unit of claim 7 further including a process controller for controlling the duration time of the whole blood processing by the coated nanoparticles, wherein chemiluminescence-generated light activates the broad-spectrum antiviral capacity of the photodynamic compounds to achieve reduction or inactivation of pathogens throughout the blood bag; and a magnet for removing nanoparticles from processed whole blood at the completion of processing.
